(19) United States
(12) Patent Application Publication
(10) Pub. No.: US 2009/0029953 A1
(43) Pub. Date: Jan. 29, 2009

(54) 18-METHYL-19-NOR-17-PREGN-4-ENE-21,17-CARBOLACTONES, AS WELL AS PHARMACEUTICAL PREPARATIONS THAT CONTAIN THE LATTER

(76) Inventors: Rolf Bohlmann, Berlin (DE); Dieter Bittler, Berlin (DE); Hermann Kuenzer, Berlin (DE); Peter Esperling, Berlin (DE); Hans Peter Muhn, Berlin (DE); Karl Heinrich Fritzemeier, Berlin (DE); Ulrike Fuhrmann, Berlin (DE); Katja Prelle, Berlin (DE); Steffen Borden, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/813,251

(22) PCT Filed: Dec. 30, 2005
(86) PCT No.: PCT/EP05/14205

§ 371 Date: May 27, 2008

(30) Foreign Application Priority Data
Dec. 30, 2004 (DE) 102004063864.0

(51) Int. Cl. A61K 31/585; C07J 21/00; A61P 15/00

(52) U.S. Cl. 514/170; 514/173; 549/265

(57) ABSTRACT

The invention relates to novel 18-methyl-19-nor-17-pregn-4-en-21,17-carbolactones of general formula (I), where Z=O, H₂, ═NOR or ═NNHSO₂R, where R=H, a straight- or branched-chain 1-4 or 3-4 C alkyl group, R⁴=H, halogen, methyl or trifluoromethyl, R⁶ and/or R⁷ can be α- or β-positioned and independently=branched- or straight-chain 1-4 or 3-4 alkyl, or R⁶=H and R⁷=a- or β-positioned, straight- or branched-chain 1-4 or 3-4 C alkyl, or R⁶ and R⁷=H, or R⁶ and R⁷ together=a- or β-positioned methylene or an additional bond. The novel compounds have gestagenic and antimineralocorticoid activity and are suitable for the production of pharmaceutical preparations, for example, for oral contraception and the treatment of pre-, peri- and post-menopausal pain.

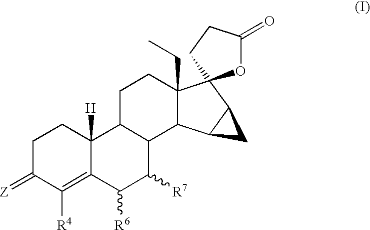

This invention relates to 18-methyl-19-nor-17-pregn-4-ene-21,17-carbolactones of general formula I

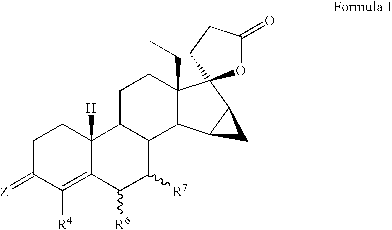

in which
Z means an oxygen atom, two hydrogen atoms, a grouping ═NOR or ═NNHSO₂R, whereby R is a hydrogen atom or a straight-chain or branched-chain alkyl group with 1 to 4 or 3 to 4 carbon atoms,
R⁴ is a hydrogen atom, a halogen atom, a methyl group, or a trifluoromethyl group,
R⁶ and/or R⁷ can be in α- or β-position, and, independently of one another, mean a straight-chain or branched-chain alkyl group with 1 to 4 or 3 to 4 carbon atoms, or
R⁶ means a hydrogen atom and R⁷ means an α- or β-position, straight-chain or branched-chain alkyl group with 1 to 4 or 3 to 4 carbon atoms, or
R⁶ and R⁷ each mean a hydrogen atom or
R⁶ and R⁷ together mean an α- or β-position methylene group or an additional bond.

Z preferably stands for an oxygen atom.

If Z stands for a grouping ═NOR or ═NNHSO₂R, R preferably is a hydrogen atom.

A methyl, ethyl, n-propyl or n-butyl group or an isopropyl, iso- or tert-butyl group can be considered for a straight-chain or branched-chain alkyl group with 1 to 4 or 3 to 4 carbon atoms.

R⁴ is preferably a hydrogen atom.

As halogen atom R⁴, a fluorine, chlorine, bromine or iodine atom is suitable; chlorine is preferred among the latter.

If R⁶ and/or R⁷ is a straight-chain or branched-chain alkyl group with 1 to 4 or 3 to 4 carbon atoms, a methyl, ethyl, n-propyl or n-butyl group or an isopropyl iso- or tert-butyl group is considered in this respect.

R⁶ and R⁷ preferably stand for a hydrogen atom and a methyl group or together for a methylene group or a double bond.

Compounds that are mentioned below are especially preferred according to the invention:
18-Methyl-15β,16β-1-methylene-3-oxo-19-nor-17-pregna-4,6-diene-21,17-carbolactone
18-Methyl-6α,7α-15β,16β-dimethylene-3-oxo-19-nor-17-pregn-4-ene-21,17-carbolactone
18-Methyl-6β,7β-15β,16β-dimethylene-3-oxo-19-nor-17-pregn-4-ene-21,17-carbolactone
7α,18-Dimethyl-15β,16β-methylene-3-oxo-19-nor-17-pregn-4-ene-21,17-carbolactone
7β,18-Dimethyl-15β,16β-methylene-3-oxo-19-nor-17-pregn-4-ene-21,17-carbolactone
3-Hydroxylamino-18-methyl-6β,7β-15β,16β-dimethylene-19-nor-17-pregn-4-ene-21,17-carbolactone
4-Chloro-18-methyl-6β,7β-15β,16β-dimethylene-3-oxo-19-nor-17-pregn-4-ene-21,17-carbolactone
7α-Ethyl-18-methyl-15β,16β-methylene-3-oxo-19-nor-17-pregn-4-ene-21,17-carbolactone
7β-Ethyl-18-methyl-15β,16β-methylene-3-oxo-19-nor-17-pregn-4-ene-21,17-carbolactone
18-Methyl-15β,16β-methylene-3-oxo-7α-propyl-19-nor-17-pregn-4-ene-21,17-carbolactone
18-Methyl-15β,16β-methylene-3-oxo-7β-propyl-19-nor-17-pregn-4-ene-21,17-carbolactone.

Drospirenone (6β,7β-15β,16β-dimethylene-3-oxo-17-pregn-4-ene-21,17β-carbolactone) is a new gestagen, which is contained in, for example, the oral contraceptive YASMIN® and the preparation ANGELIQ® for treating post-menopausal symptoms (both SCHERING AG). Based on its comparatively low affinity to the gestagen receptor and its comparatively high ovulation-inhibiting dose,

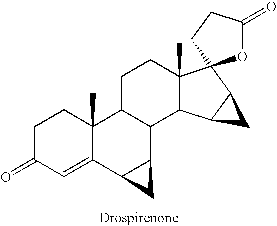

drospirenone in YASMIN® is contained in the relatively high daily dose of 3 mg. Drospirenone is characterized in that in addition to the gestagenic action, it has aldosterone-antagonistic (anti-mineral-corticoid) as well as antiandrogenic action. These two properties make the drospirenone very similar in its pharmacological profile to the natural gestagen progesterone, which, however, unlike drospirenone, is not sufficiently orally bio-available.

It is therefore the object of this invention to make available compounds that have a stronger bond to the gestagen receptor than the drospirenone and thus are to have a higher gestagenic power than the drospirenone. This is ultimately to be displayed in a lower daily dosage and is to result in a lower substance demand in the active compound.

This object is achieved by the preparation of the 18-methyl-19-nor-17-pregn-4-ene-21,17-carbolactones of general formula I that are described here. The compounds of general formula I (and in particular compound 3b, see experimental part) can be regarded as the constitutional isomers of the drospirenone. The new compounds are distinguished in the gestagen binding test with use of cytosol from the rabbit uterus homogenate and 3H-progesterone as a reference substance through a higher affinity to the gestagen receptor than drospirenone and through comparable affinity to the mineral corticoid receptor from the rat kidney homogenate.

The binding to the gestagen receptor is in this case, surprisingly enough, up to 5× as strong as that of the drospirenone.

The compounds according to the invention are distinguished, surprisingly enough, by strong gestagenic action and are greatly effective in the pregnancy-maintenance test in rats after subcutaneous administration.

### Executing the Pregnancy Maintenance Test in Rats:

In pregnant rats, removal of the corpora lutea or castration induces abortion. By exogenous supplying of progestins (gestagens) in combination with a suitable dose of estrogen, pregnancy can be maintained. The pregnancy maintenance test in ovariectomized rats serves to determine peripheral gestagenic activity of a compound.

Rats are paired up during the proestrus overnight. Mating is monitored on the morning of the following day by vaginal smear inspection. The presence of sperm is considered in this case as day 1 of the beginning of pregnancy. On day 8 of pregnancy, the animals are ovariectomized under ether anesthesia. Treatment with the test compound and exogenous estrogen (estrone, 5 μg/kg/day) is performed subcutaneously once daily on day 8 to day 15 or day 21 of pregnancy. The first administration on day 8 is performed 2 hours prior to castration. Intact control animals receive only the vehicle.

### Evaluation:

At the end of the test (day 15 or day 21), the animals are sacrificed under CO₂ atmosphere, and live fetuses (fetuses with beating hearts) and implantation sites (early resorptions and dead fetuses including autolysis and atrophic placentas) in both uterine horns are counted. In addition, on day 22, fetuses can be inspected for malformations. In uteri without fetuses or implantation sites, the number of nidation sites is determined by staining with 10% ammonium sulfide solution. The pregnancy maintenance rate is calculated as the quotient of the number of living fetuses and the total number of nidation sites (both resorbed and dead fetuses as well as nidation sites). In this case, for the test substance 18-methyl-6α,7α-15β,16β-dimethylene-3-oxo-19-nor-17-pregn-4-ene-21,17-carbolactone (compound 3a, see the experimental section), a pregnancy-maintaining dose (ED₅₀) of 120 μg/kg/day was determined. For drospirenone, this value is 3.5 mg/kg/day.

The compounds of general formula I according to the invention have very strong gestagenic action with a simultaneously weaker binding to the androgen receptor (dissociation).

In addition, it was found that compounds according to the invention show a potassium-retaining, natriuretic (anti-mineral-corticoid) action in adrenalectomized rats.

Based on their gestagenic action, the new compounds of general formula I can be used by themselves or in combination with estrogen in pharmaceutical preparations for contraception.

Because of their advantageous profile of action, the compounds according to the invention are especially well suited for treatment of premenstrual symptoms, such as headaches, depressive mood disorders, water retention and mastodynia.

The dosage of the compounds according to the invention in contraceptive preparations is to be 0.01 to 5 mg, preferably 0.01 to 2 mg per day.

The daily dose in the treatment of premenstrual symptoms is approximately 0.1 to 20 mg.

The gestagenic and estrogenic active ingredient components are preferably administered orally together in contraceptive preparations. The daily dose is preferably administered once.

As estrogens, synthetic estrogens, preferably ethinyl estradiol, but also mestranol, are considered.

The estrogen is administered in a daily amount that corresponds to that of 0.01 to 0.04 mg of ethinyl estradiol.

The new compounds of general formula I can also be used in pharmaceutical preparations for treating pre-, peri- and post-menopausal symptoms, as well as in preparations for hormone substitution therapy (HRT).

As estrogens in such preparations, primarily natural estrogens, mainly estradiol or its esters, for example estradiol valerate or else conjugated estrogens (CEEs=conjugated equine estrogens), as they are contained in, for example, the preparation PREMARIN®, are used.

The formulation of the pharmaceutical preparations based on the new compounds is carried out in a way that is known in the art by the active ingredient, optionally in combination with an estrogen, being processed with the vehicles, diluents, optionally flavoring correctives, etc., that are commonly used in galenicals and being converted into the desired form of administration.

For the preferred oral administration, in particular tablets, coated tablets, capsules, pills, suspensions or solutions are suitable.

For parenteral administration, in particular oily solutions, such as, for example, solutions in sesame oil, castor oil and cottonseed oil, are suitable. To increase the solubility, solubilizers, such as, for example, benzyl benzoate or benzyl alcohol, can be added.

It is also possible to incorporate the substances according to the invention into a transdermal system and thus to administer them transdermally.

The new compounds of general formula I are produced as described below according to the invention. The synthesis route for the novel 18-methyl-6,7-15,16-dimethylene-3-oxo-19-nor-17-pregn-4-ene-21,17-carbolactones according to Diagram 1 starts in the example of compound 1 (DE 3402329).

The introduction of a Δ⁶-double bond is carried out with, for example, bromination of 3,5-dienamine or by a modified dienol ether bromination as well as subsequent hydrogen bromide cleavage (see, e.g., J. Fried, J. A. Edwards, Organic Reactions in Steroid Chemistry, von Nostrand Reinhold Company 1972, pp. 265-374).

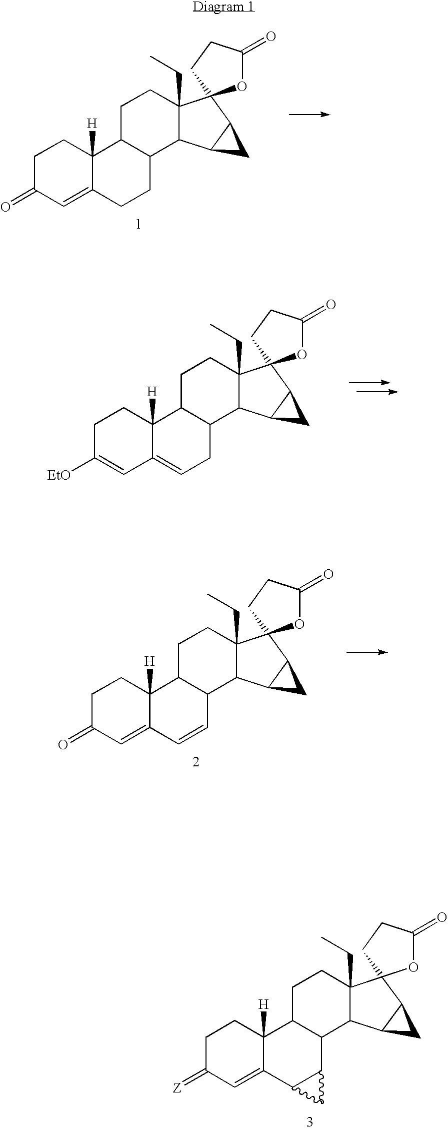

The dienol ether bromination can be carried out, e.g., analogously to the instructions of J. A. Zderie, Humberto Carpio, A. Bowers and Carl Djerassi in Steroids 1, 233 (1963). The hydrogen bromide cleavage can be accomplished by heating the 6-bromine compound with basic reagents, such as, e.g., LiBr or Li₂CO₃, in aprotic solvents, such as dimethylformamide, at temperatures of 50-120° C. or else by the 6-bromine compounds in a solvent such as collidine or lutidine being heated to compound 2 (Example 1).

Compound 2 is then converted into a compound 3 by methylenation of the Δ⁶-double bond according to known processes, e.g., with dimethyl sulfoxonium methylide (see, e.g., DE-A 11 83 500, DE-A 29 22 500, EP-A 0 019 690, U.S. Pat. No. 4,291,029; E. J. Corey and M. Chaykovsky, J. Am. Chem. Soc. 84, 867 (1962)), whereby a mixture of α- and β-isomers (compounds 3a/3b) is obtained (the ratio is dependent on the substrates used and is approximately 1:1), which can be separated into the individual isomers by, e.g., chromatography.

The introduction of a substituent R⁴ can be handled, for example, starting from a compound of formula 3 by epoxidation of the Δ⁴-double bond with hydrogen peroxide under alkaline conditions and reaction of the epoxides that are produced in a suitable solvent with acids of general formula H—R⁴, whereby —R⁴ can be a halogen atom or a pseudohalogen, or is reacted with catalytic amounts of mineral acid, and optionally the 4-bromine compounds of general formula I that are obtained (whereby R⁴=bromine) are reacted with 2,2-difluoro-2-(fluorosulfonyl)acetic acid methyl ester in dimethylformamide in the presence of copper(I) iodide.

The introduction of a 6-methylene group can be carried out, for example, starting from a 3-amino-3,5-diene derivative by reaction with formalin in alcoholic solution with the formation of a 6α-hydroxymethyl group and subsequent acidic dehydration with, e.g., hydrochloric acid in dioxane/water. The dehydration, however, can also be carried out in the way that first the hydroxy group is exchanged for a better leaving group and then eliminated. As leaving groups, e.g., the mesylate, tosylate or benzoate is suitable (see DE-A 34 02 3291, EP-A. 0 150 157, U.S. Pat. No. 4,584,288; K. Nickisch et al., J. Med. Chem. 34, 2464 (1991)).

Another possibility for the production of 6-methylene compounds exists in the direct reaction of 4(5)-unsaturated 3-ketones with acetalene of the formaldehyde in the presence of sodium acetate with, e.g., phosphorus oxychloride or phosphorus pentachloride in suitable solvents such as chloroform (see, e.g., K. Annen, H. Hofmeister, H. Laurent and R. Wiechert, Synthesis 34 (1982)).

The 6-methylene compounds can be used for the production of compounds of general formula I, in which R⁶ is equal to methyl, and R⁶ and R⁷ together form an additional bond.

To this end, there can be used, e.g., a process that is described by D. Burn et al. in Tetrahedron 21, 1619 (1965) in which an isomerization of the double bond can be achieved by heating the 6-methylene compounds in ethanol with 5% palladium-carbon catalyst, which was pretreated either with hydrogen or by heating with a small amount of cyclohexene. The isomerization can also be carried out with a non-pretreated catalyst, if a small amount of cyclohexene is added to the reaction mixture. The occurrence of small proportions of hydrogenated products can be prevented by adding excess sodium acetate.

The production of 6-methyl-4,6-dien-3-one derivatives can also be carried out directly, however (see K. Annen, H. Hofmeister, H. Laurent and R. Wiechert, Lieb. Ann. 712 (1983)).

Compounds in which R⁶ represents an α-methyl function can be produced from the 6-methylene compounds by hydrogenation under suitable conditions. The best results (selective hydrogenation of the exo-methylene function) are achieved by transfer hydrogenation (E. A. Brande, R. P. Linstead and P. W. D. Mitchell, J. Chem. Soc. 3578 (1954)). If the 6-methylene derivatives are heated in a suitable solvent, such as, e.g., ethanol, in the presence of a hydride donor, such as, e.g., cyclohexene, very good yields of 6α-methyl derivatives thus result. Small portions of 6β-methyl compound can be isomerized in acidic form (see, e.g., D. Burn, D. N. Kirk and V. Petrow, Tetrahedron 1619 (1965)).

The specific production of 6β-alkyl compounds is also possible. In this respect, the 4(5)-unsaturated 3-ketones are reacted with, e.g., ethylene glycol, trimethyl orthoformate in dichloromethane in the presence of catalytic amounts of an acid (e.g., p-toluenesulfonic acid) to form the corresponding 3-ketals. During this ketalization, the double bond isomerizes in 5(6)-position. A selective epoxidation of this 5(6)-double bond can be accomplished by, e.g., using organic peracids, e.g., m-chloroperbenzoic acid, in suitable solvents such as dichloromethane. As an alternative to this, the epoxidation can also be carried out with hydrogen peroxide in the presence of, e.g., hexachloroacetone or 3-nitrotrifluoroacetophenone. The 5,6α-epoxides that are formed can then be opened axially with use of corresponding alkylmagnesium halides or alkyl lithium compounds. 5α-Hydroxy-6β-alkyl compounds are thus obtained. The cleavage of the 3-keto protective group can be carried out while obtaining the 5α-hydroxy function by treatment under mild acidic conditions (acetic acid or 4N hydrochloric acid at 0° C.). Basic elimination of the 5α-hydroxy function with, e.g., dilute aqueous sodium hydroxide solution yields the 3-keto-4-ene compounds with a deposition 6-alkyl group. As an alternative to this, the ketal cleavage under more drastic conditions (aqueous hydrochloric acid or another strong acid) yields the corresponding 6α-alkyl compounds.

The compounds of general formula I that are obtained, in which Z stands for an oxygen atom, can optionally be converted by reaction with hydroxylamine hydrochloride in the presence of a tertiary amine at temperatures of between −20 and +40° C. into their corresponding oximes (general formula I with Z in the meaning of ═NOH, whereby the hydroxy group can be in syn- or anti-position). Suitable tertiary bases are, for example, trimethylamine, triethylamine, pyridine, N,N-dimethylaminopyridine, 1,5-diazabicyclo[4.3.0]non-5-ene (DBN) and 1,5-diazabicyclo[5.4.0]undec-5-ene (DBU), whereby pyridine is preferred. This is analogous to the description in WO 98/24801 for the production of corresponding 3-oxyimino derivatives of the drospirenone.

The removal of the 3-oxo group for the production of an end product of general formula I with Z in the meaning of two hydrogen atoms can be carried out, for example, according to the instructions that are indicated in DE-A 28 05 490 by reductive cleavage of a thioketal of the 3-keto compound.

The examples below are used for a more detailed explanation of the invention:

### EXAMPLE 1

### 18-Methyl-15β,16β-methylene-3-oxo-19-nor-17-pregna-4,6-diene-21,17-carbolactone

11 ml of o-formic acid triethyl ester as well as 11 ml of dioxane/sulfuric acid (12+0.42) are added to a solution of 11.0 g of 18-methyl-15β,16β-methylene-3-oxo-19-nor-17-pregn-4-ene-21,17-carbolactone (DE3402329) in 110 ml of dioxane. 14.4 g of 3-ethoxy-18-methyl-15β,16β-methylene-19-nor-17-pregna-3,5-diene-21,17-carbolactone was obtained as a crude product. The latter was dissolved in 380 ml of acetone, mixed with 2.3 ml of pyridine, 10.3 g of sodium acetate, 28 ml of water as well as 7.6 g of N-bromosuccinimide, and it was stirred for 0.5 hour at ice bath temperature. Then, it was stirred into ice water, the precipitate was filtered out, it was washed with water, the precipitate was taken up in dichloromethane, washed with water, dried on sodium sulfate and concentrated by evaporation in a vacuum. 17 g of 6-bromo-18-methyl-3-oxo-15β,16β-methylene-19-nor-17-pregn-4-ene-21,17-carbolactone was obtained as a crude product. The latter was dissolved in 170 ml of dimethylformamide and stirred with 6.65 g of lithium bromide as well as 7.87 g of lithium carbonate for one hour at 100° C. Then, it was stirred into ice water, the precipitate was filtered out, washed with water, the precipitate was taken up in dichloromethane, washed with water, dried on sodium sulfate and concentrated by evaporation in a vacuum. After chromatography on silica gel with hexane/acetone, 6.5 g of 18-methyl-15β,16β-methylene-3-oxo-19-nor-17-pregna-4,6-diene-21,17-carbolactone (compound 2 in Diagram 1) with a melting point of 187° C. is obtained.

### EXAMPLE 2

### 18-Methyl-6α,7α-15β,16β-dimethylene-3-oxo-19-nor-17-pregn-4-ene-21,17-carbolactone

A solution of 5.8 g of 18-methyl-153,161-methylene-3-oxo-19-nor-17-pregna-4,6-diene-21,17-carbolactone in 200 ml of dimethyl sulfoxide was mixed with 9.06 g of trimethylsulfoxonium iodide and 1.613 g of sodium hydride (55% suspension in oil), and it was stirred for 20 hours under argon at room temperature. Then, it was stirred into ice water, turned weakly acidic, the precipitate was filtered out, the precipitate was taken up in dichloromethane, washed with water, dried on sodium sulfate, concentrated by evaporation in a vacuum, and chromatographed on silica get with hexane/acetone. After recrystallization of fraction III from 2-propanol/acetone, 0.8 g of 18-methyl-6α,7α-15β,16β-dimethylene-3-oxo-19-nor-17-pregn-4-ene-21,17-carbolactone (compound 3a) was obtained as crystals with a melting point of 262° C. [α]_{D}+89.7° (methanol, c=10.15 mg/ml)

### EXAMPLE 3

### 18-Methyl-6β,7β-15β,16β-dimethylene-3-oxo-19-nor-17-pregn-4-ene-21,17-carbolactone

According to the method of Example 2, 0.9 g of 18-methyl-6β,7β-15β,16β-dimethylene-3-oxo-19-nor-17-pregn-4-ene-21,17-carbolactone (compound 3b) was obtained after the chromatography as fraction IV as a solid with a melting point of 189-190° C. [α]_{D}=−121.4° (chloroform, c=10.7 mg/ml) and [α]_{D}=+137.9° (methanol, c=10.63 mg/ml)

### EXAMPLE 4

### 7α,18-Dimethyl-15β,16β-methylene-3-oxo-19-nor-17-pregn-4-ene-21,17-carbolactone

In a solution of 0.4 g of 18-methyl-15β,16β-methylene-3-oxo-19-nor-17-pregna-4,6-diene-21,17-carbolactone in 7 ml of tetrahydrofuran, 12 mg of copper(I) chloride was added at room temperature and stirred for 10 minutes before it was cooled to −15° C., mixed with 75 mg of aluminum chloride, stirred for 30 minutes at this temperature, mixed drop by drop with 0.8 ml of methylmagnesium bromide solution (3 M in diethyl ether), and stirred for one hour at −10° C. For working-up, the reaction mixture was mixed with 4N hydrochloric acid at −10° C., stirred for 1.5 hours at room temperature, added to water, extracted three times with ethyl acetate, dried on sodium sulfate, concentrated by evaporation in a vacuum and chromatographed on silica gel with hexane/ethyl acetate. After recrystallization of fraction 1,182 mg of 7α,18-dimethyl-15β,16β-methylene-3-oxo-19-nor-17-pregn-4-ene-21,17-carbolactone as crystals with a melting point of 250° C. was obtained. [α]_{D}=53.1+/−0.3° (chloroform, c=10.3 mg/ml)

### EXAMPLE 5

### 7β,18-Dimethyl-15β,16β-methylene-3-oxo-19-nor-17-pregn-4-ene-21,17-carbolactone

According to the method of Example 4, after chromatography as fraction II, 36 mg of 7β,18-dimethyl-15β,16β-methylene-3-oxo-19-nor-17-pregn-4-ene-21,17-carbolactone was obtained as a solid with a melting point of 226° C. [α]_{D}=9.0+/−0.5° (chloroform, c=10.2 mg/ml).

### EXAMPLE 6

### 7α-Ethyl-18-methyl-15β,16β-methylene-3-oxo-19-nor-17-pregn-4-ene-21,17-carbolactone

15 mg of copper(I) chloride was added at room temperature to a solution of 0.5 g of 18-methyl-15β,16β-methylene-3-oxo-19-nor-17-pregna-4,6-diene-21,17-carbolactone in 10 ml of tetrahydrofuran, and it was stirred for 10 minutes before it was cooled to −15° C., mixed with 93 mg of aluminum chloride, stirred for 30 minutes at this temperature, mixed drop by drop with 1.0 ml of ethyl magnesium bromide solution (3 M in diethyl ether), and stirred for one hour at −110° C. For working-up, the reaction mixture was mixed with 3 ml of 2N hydrochloric acid at −10° C., stirred for 0.5 hour at room temperature, poured into water, extracted three times with ethyl acetate, dried on sodium sulfate, concentrated by evaporation in a vacuum, and chromatographed on silica gel with hexane/ethyl acetate. After recrystallization of fraction 1,180 mg of 7α-ethyl-18-methyl-15β,16β-methylene-3-oxo-19-nor-17-pregn-4-ene-21,17-carbolactone was obtained as crystals with a melting point of 205-208° C. [α]_{D}=+34.4+/−0.3° (chloroform, c=10.3 mg/ml)

### EXAMPLE 7

### 7β-Ethyl-18-methyl-15β,16β-methylene-3-oxo-19-nor-17-pregn-4-ene-21,17-carbolactone

According to the method of Example 6, after chromatography as fraction II, 110 mg of 7β-ethyl-18-methyl-15β,16β-methylene-3-oxo-19-nor-17-pregn-4-ene-21,17-carbolactone was obtained as a solid with a melting point of 169-171° C. [α]_{D}=+30.8+/−0.5° (chloroform, c=10.1 mg/ml).

### EXAMPLE 8

### 18-Methyl-15β,16β-methylene-3-oxo-7α-propyl-19-nor-17-pregn-4-ene-21,17-carbolactone

15 mg of copper(I) chloride was added at room temperature to a solution of 0.5 g of 18-methyl-15β,16β-methylene-3-oxo-19-nor-17-pregna-4,6-diene-21,17-carbolactone in 10 ml of tetrahydrofuran, and it was stirred for 10 minutes before it was cooled to −15° C., mixed with 93 mg of aluminum chloride, stirred for 30 minutes at this temperature, mixed drop by drop with 1.5 ml of propylmagnesium bromide solution (2 M in tetrahydrofuran) and stirred for one hour at −10° C. For working-up, the reaction mixture was mixed with 3 ml of 2N hydrochloric acid at −10° C., stirred for 0.5 hour at room temperature, poured into water, extracted three times with ethyl acetate, dried on sodium sulfate, concentrated by evaporation in a vacuum and chromatographed on silica gel with hexane/ethyl acetate. After recrystallization of fraction I, 177 mg of 18-methyl-15β,16β-methylene-3-oxo-7α-propyl-19-nor-17-pregn-4-ene-21,17-carbolactone was obtained as crystals with a melting point of 167.5° C. [α]_{D}=+31.2+/−0.3° (chloroform, c=10.1 mg/ml)

### EXAMPLE 9

### 18-Methyl-15β,16β-methylene-3-oxo-7β-propyl-19-nor-17-pregn-4-ene-21,17-carbolactone

According to the method of Example 8, after chromatography as fraction II, 105 mg of 18-methyl-15β,16β-methylene-3-oxo-7β-propyl-19-nor-17-pregn-4-ene-21,17-carbolactone was obtained as a solid with a melting point of 90.9° C. [α]_{D}+27.4+/−0.4° (chloroform, c=10.3 mg/ml).

1. 18-Methyl-19-nor-17-pregn-4-ene-21,17-carbolactones of general formula I 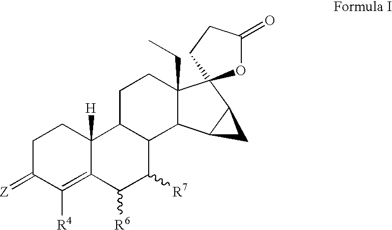
in which
Z means an oxygen atom, two hydrogen atoms, a grouping ═NOR or ═NNHSO₂R, whereby R is a hydrogen atom or a straight-chain or branched-chain alkyl group with 1 to 4 or 3 to 4 carbon atoms,
R⁴ is a hydrogen atom, a halogen atom, a methyl group or a trifluoromethyl group,
R⁶ and/or R⁷ can be in α- or β-position, and, independently of one
another, mean a straight-chain or branched-chain alkyl group with 1 to 4 or 3 to 4 carbon atoms, or
R⁶ means a hydrogen atom, and R⁷ means an α- or β-position, straight-chain or branched-chain alkyl group with 1 to 4 or 3 to 4 carbon atoms, or
R⁶ and R⁷ each mean a hydrogen atom, or
R⁶ and R⁷ together mean an α- or β-position methylene group or an additional bond.

2. Compounds of general formula I according to claim 1, in which Z stands for an oxygen atom.

3. Compounds of general formula I according to claim 1, in which Z stands for a grouping ═NOR or ═NNHSO₂R, and R therein stands for a hydrogen atom.

4. Compounds of general formula I according to claim 1, in which R⁴ is a hydrogen atom.

5. Compounds of general formula I according to claim 1, in which R⁴ is a chlorine atom.

6. Compounds of general formula I according to claim 1, in which R⁶ stands for a hydrogen atom, and R⁷ stands for a methyl group.

7. Compounds of general formula I according to claim 1, in which R⁶ stands for a hydrogen atom and R⁷ stands for a propyl group.

8. Compounds of general formula I according to claim 1, in which R⁶ and R⁷ together stand for a methylene group.

9. Compounds of general formula I according to claim 1, in which R⁶ and R⁷ together stand for a double bond.

10. Compounds of general formula I according to claim 1, namely
18-Methyl-15β,16β-methylene-3-oxo-19-nor-17-pregna-4,6-diene-21,17-carbolactone
18-Methyl-6α,7α-15β,16β-dimethylene-3-oxo-19-nor-17-pregn-4-ene-21,17-carbolactone
18-Methyl-6β,7β-15β,16β-dimethylene-3-oxo-19-nor-17-pregn-4-ene-21,17-carbolactone
7α,18-Dimethyl-15β,16β-methylene-3-oxo-19-nor-17-pregn-4-ene-21,17-carbolactone
7β,18-Dimethyl-15β,16β-methylene-3-oxo-19-nor-17-pregn-4-ene-21,17-carbolactone
3-Hydroxylamino-18-methyl-6β,7β-15β,16β-dimethylene-19-nor-17-pregn-4-ene-21,17-carbolactone
4-Chloro-18-methyl-6β,7β-15β,16β-dimethylene-3-oxo-19-nor-17-pregn-4-ene-21,17-carbolactone
7α-Ethyl-18-methyl-15β,16β-methylene-3-oxo-19-nor-17-pregn-4-ene-21,17-carbolactone
7α-Ethyl-18-methyl-15β,16β-methylene-3-oxo-19-nor-17-pregn-4-ene-21,17-carbolactone
18-Methyl-15β,16β-methylene-3-oxo-7α-propyl-19-nor-17-pregn-4-ene-21,17-carbolactone
18-Methyl-15β,16β-methylene-3-oxo-7β-propyl-19-nor-17-pregn-4-ene-21,17-carbolactone.

11. Pharmaceutical preparations that contain at least one compound according to claim 1 as well as a pharmaceutically harmless vehicle.

12. Pharmaceutical preparations according to claim 11, in addition containing at least one estrogen.

13. Pharmaceutical preparations according to claim 12, containing ethinyl estradiol.

14. Pharmaceutical preparations according to claim 12, containing a natural estrogen.

15. Pharmaceutical preparations according to claim 14, containing estradiol.

16. Pharmaceutical preparations according to claim 14, containing estradiol valerate.

17. Pharmaceutical preparations according to claim 14, containing at least one conjugated estrogen.

18. A method of achieving oral contraception comprising administering a compound of claim 1.
